SEQUENCE LISTING (3 sequences)

SEQ ID NO: 1 - protein, 425 aa, Homo sapiens
MEPSATPGAQMGVPPGSREPSPVPPDYEDEFLRYLWRDYLYPKQYEWVLIAAYVAVFVVALVGNTLVCLAVWRNHHMRTVTNYFIVNLSLADVLVTAICLPASLLVDITESWLFGHALCKVIPYLQAVSVSVAVLTLSFIALDRWYAICHPLLFKSTARRARGSILGIWAVSLAIMVPQAAVMECSSVLPELANRTRLFSVCDERWADDLYPKIYHSCFFIVTYLAPLGLMAMAYFQIFRKLWGRQIPGTTSALVRNWKRPSDQLGDLEQGLSGEPQPRGRAFLAEVKQMRARRKTAKMLMVVLLVFALCYLPISVLNVLKRVFGMFRQASDREAVYACFTFSHWLVYANSAANPIIYNFLSGKFREQFKAAFSCCLPGLGPCGSLKAPSPRSSASHKSLSLQSRCSISKISEHVVLTSVTTVLP

SEQ ID NO: 2 - protein, 34 aa, Homo sapiens
  MISC_FEATURE (1)..(1): X is pyruvoglutamate
XPLPDCCRQKTCSCRLYELLHGAGNHAAGILTLX

SEQ ID NO: 3 - protein, 28 aa, Homo sapiens
FSGPPGLQGRLQRLLQASGNHAAGILTM

(19) United States
(12) Patent Application Publication
(10) Pub. No.: US 2015/0140015 A1
(43) Pub. Date: May 21, 2015

(54) METHODS AND PHARMACEUTICAL COMPOSITIONS FOR THE TREATMENT OF PANCREATIC CANCER

(71) Applicant: INSERM (Institut National de la Sante et de la Recherche Medicale), Paris (FR)

(72) Inventor: Thierry VOISON, Paris (FR)

(21) Appl. No.: 14/600,150

(22) Filed: Jan. 20, 2015

Related U.S. Application Data
(63) Continuation-in-part of application No. PCT/FR2013/052753, filed on Nov. 15, 2013.

(51) Int. Cl. A61K 38/22; C07K 16/28; C12N 15/115; G01N 33/574; A61K 31/7088; A61K 39/395; G01N 33/50; A61K 38/17; A61K 45/06

(52) U.S. Cl. CPC A61K 38/22; A61K 38/17; C07K 16/28; C12N 15/115; A61K 45/06; A61K 31/7088; A61K 39/39558; G01N 33/5011; G01N 33/57492; C07K 2317/75; A61K 2039/505
USPC 424/172.1; 514/19.3; 514/44 R; 514/18.1; 506/10; 506/9

(57) ABSTRACT

The present invention relates to methods and pharmaceutical compositions for the treatment of pancreatic cancers. In particular, the present invention relates to an OX1R agonist for use in the treatment of pancreatic cancer in a subject in need thereof.

### FIELD OF THE INVENTION

The present invention relates to methods and pharmaceutical compositions for the treatment of pancreatic cancers.

### BACKGROUND OF THE INVENTION

Pancreatic cancer is an aggressive disease associated with an extremely poor prognosis. It is one of the most malignant cancers, characterized insidious onset, usually late diagnosis and low survival rate after diagnosis. For example, pancreatic ductal adenocarcinoma (PDAC) is the fourth leading cause of cancer death in the United States. In spite of recent therapeutic advances, long term survival in PDAC is often limited to patients who have had surgery in early stage of the disease. The biological aggressiveness of PDAC is due, in part, to the tumor's resistance to chemotherapy. Presently, the standard of treatment remains systemic chemotherapy with gemcitabine, with palliative objectives and a disappointing marginal survival advantage. Very recently, the demonstration of a clinically and statistically meaningful survival advantage with the 5-fluorouracil, leucovorin, irinotecan and oxaliplatin (FOLFIRINOX) regimen over single-agent gemcitabine (Conroy et al., N. Engl. J. Med., 364: 1817-1825 (2011)), and the introduction of nanoparticles of albumin-bound paclitaxel (nad-paclitaxel) to putatively target the desmoplastic stroma characteristic of pancreatic ductal adenocarcinoma (PDAC) (Garber, K., J. Natl. Cancer Inst., 102: 448-450 (2010)), have raised hope that innovative combinations and improved delivery of classical cytotoxics may indeed substantially affect chemotherapy efficacy in advanced PDAC. Therefore, despite marginal advances in pancreatic cancer treatment, there remains a need for improved therapies and more creative approaches to devising and delivering effective pancreatic cancer therapies.

The orexins (hypocretins) comprise two neuropeptides produced in the hypothalamus: the orexin A (OX-A) (a 33 amino acid peptide) and the orexin B (OX-B) (a 28 amino acid peptide) (Sakurai T. et al., Cell, 1998, 92, 573-585). Orexins are found to stimulate food consumption in rats suggesting a physiological role for these peptides as mediators in the central feedback mechanism that regulates feeding behaviour. Orexins regulate states of sleep and wakefulness opening potentially novel therapeutic approaches for narcoleptic or insomniac patients. Orexins have also been indicated as playing a role in arousal, reward, learning and memory. Two orexin receptors have been cloned and characterized in mammals. They belong to the super family of G-protein coupled receptors (7-transmembrane spanning receptor) (Sakurai T. et al., Cell, 1998, 92, 573-585): the orexin-1 receptor (OX1R or HCTR1) is selective for OX-A and the orexin-2 receptor (OX2R or HCTR2) is capable to bind OX-A as well as OX-B. A recent study shows that activation of OX1R by orexin can promote robust in vitro and in vivo apoptosis in colon cancer cells even when they are resistant to the most commonly used drug in colon cancer chemotherapy (Voisin T, El Firar A, Fasseu M, Rouyer-Fessard C, Descatoire V, Walker F, Paradis V, Bedossa P, Henin D, Lehy T, Laburthe M. Aberrant expression of OX1 receptors for orexins in colon cancers and liver metastases: an openable gate to apoptosis. Cancer Res. 2011 May 1; 71(9):3341-51). Remarkably, all primary colorectal tumors regardless of their localization and Duke's stages expressed OX1R while adjacent normal colonocytes as well as control normal tissues were negative. Thus this study supports that OX1R is an Achilles's heel of colon cancers (even chemoresistance) and suggests that OX1R agonists might be novel candidates for colon cancer therapy.

### SUMMARY OF THE INVENTION

Surprisingly, in addition to being expressed in colorectal cancer cells but not in normal colon cells, OX1R is also expressed in pancreatic cancer cells but not in normal pancreatic cells. Further, when cancer cells expressing OX1R are contacted with an OX1R agonist, the cells undergo apoptosis. This discovery permits the development and use of OX1R agonists to selectively kill pancreatic cancer cells while leaving normal cells (e.g. normal, non-cancerous pancreatic cells) alive. That is, from among pancreatic cells, only those which express OX1R, i.e. only cells which are cancerous, are killed, while normal pancreatic cells or other normal cells are not killed.

Accordingly, the present invention relates to methods and pharmaceutical compositions for the treatment of pancreatic cancers. In particular, the present invention relates to an OX1R agonist for use in the treatment of pancreatic cancer in a subject in need thereof.

In certain aspects, the disclosure provides methods of selectively killing pancreatic cancer cells, the methods comprising contacting the pancreatic cancer cells with amount of an OX1R agonist that is sufficient to cause apoptosis of said pancreatic cancer cells. The method is advantageously selective in that exposure to the OX1R agonist does not cause apoptosis in (and hence the death of) normal, non-cancer cells, since the pancreatic cancer cells express OX1R and the normal cells do not express OX1R.

In further aspects, the disclosure provides methods of decreasing the size of an established pancreatic cancer tumor in a patient in need thereof, comprising administering to the patient a therapeutically effective amount of an OX1R agonist.

In other aspects, the disclosure provides methods of preventing or slowing tumor growth in a patient in need thereof, comprising administering to said patient a therapeutically effective amount of an OX1R agonist.

### DETAILED DESCRIPTION OF THE INVENTION

The present invention relates to an OX1R agonist for use in the treatment of pancreatic cancer in a subject in need thereof.

As used herein, the term “OX1R” has its general meaning in the art and refers to the 7-transmembrane spanning receptor OX1R for orexins. According to the invention, OX1R promotes apoptosis in the human prancreatic cancer cell line through a mechanism which is not related to Gq-mediated phopholipase C activation and cellular calcium transients. Orexins induce indeed tyrosine phosphorylation of 2 tyrosine-based motifs in OX1R, ITIM and ITSM, resulting in the recruitment of the phosphotyrosine phosphatase SHP-2, the activation of which is responsible for mitochondrial apoptosis (Voisin T, El Firar A, Rouyer-Fessard C, Gratio V, Laburthe M. A hallmark of immunoreceptor, the tyrosine-based inhibitory motif ITIM, is present in the G protein-coupled receptor OX1R for orexins and drives apoptosis: a novel mechanism. FASEB J. 2008 June; 22(6):1993-2002.; El Firar A, Voisin T, Rouyer-Fessard C, Ostuni M A, Couvineau A, Laburthe M. Discovery of a functional immunoreceptor tyrosine-based switch motif in a 7-transmembrane-spanning receptor: role in the orexin receptor OX1R-driven apoptosis. FASEB J. 2009 December; 23(12):4069-80. doi: 10.10964109-131367. Epub 2009 Aug. 6). An exemplary amino acid sequence of OX1R is shown as SEQ ID NO:1.

orexin receptor-1 OX1R
(SEQ ID NO: 1)

| | |
|---|---|
| 1 | mepsatpgaq mgvppgsrep spvppdyede flrylwrdyl |
| | ypkqyewvli aayvavfvva |
| 61 | lvgntivcla vwrnhhmrtv tnyfivnlsl advlvtaicl |
| | pasllvdite swlfghalck |
| 121 | vipylqaysv svavltlsfi aldrwyaich pllfkstarr |
| | argsilgiwa vslaimvpqa |
| 181 | avmecssvlp elanrtrlfs vcderwaddl ypkiyhscff |
| | ivtylaplgl mamayfqifr |
| 241 | klwgrqipgt tsalvrnwkr psdqlgdleq glsgepqprg |
| | raflaevkqm rarrktakml |
| 301 | mvvllvfalc ylpisvinvl kryfgmfrqa sdreavyacf |
| | tfshwlvyan saanpiiynf |
| 361 | lsgkfreqfk aafscclpgl gpcgslkaps prssashksl |
| | slqsrcsisk isehvvltsv |
| 421 | ttvlp |

Accordingly, as used herein, the term “OX1R agonist” refers to any compound natural or not that is able to bind to OX1R and promotes OX1R activity which consists of activation of signal transduction pathways involving recruitment of SHP-2 and the induction of apoptosis of the cell, independently of transient calcium release.

In some embodiments, the OX1R agonist is a small organic molecule. The term “small organic molecule” refers to a molecule of a size comparable to those organic molecules generally used in pharmaceuticals. The term excludes biological macromolecules (e. g., proteins, nucleic acids, etc.). Preferred small organic molecules range in size up to about 5000 Da, more In particular up to 2000 Da, and most In particular up to about 1000 Da.

In some embodiment, the OX1R agonist is an OX1R antibody or a portion thereof.

As used herein, “antibody” includes both naturally occurring and non-naturally occurring antibodies. Specifically, “antibody” includes polyclonal and monoclonal antibodies, and monovalent and divalent fragments thereof. Furthermore, “antibody” includes chimeric antibodies, wholly synthetic antibodies, single chain antibodies, and fragments thereof. The antibody may be a human or nonhuman antibody. A nonhuman antibody may be humanized by recombinant methods to reduce its immunogenicity in man.

In one embodiment of the antibodies or portions thereof described herein, the antibody is a monoclonal antibody. In one embodiment of the antibodies or portions thereof described herein, the antibody is a polyclonal antibody. In one embodiment of the antibodies or portions thereof described herein, the antibody is a humanized antibody. In one embodiment of the antibodies or portions thereof described herein, the antibody is a chimeric antibody. In one embodiment of the antibodies or portions thereof described herein, the portion of the antibody comprises a light chain of the antibody. In one embodiment of the antibodies or portions thereof described herein, the portion of the antibody comprises a heavy chain of the antibody. In one embodiment of the antibodies or portions thereof described herein, the portion of the antibody comprises a Fab portion of the antibody. In one embodiment of the antibodies or portions thereof described herein, the portion of the antibody comprises a F(ab′)2 portion of the antibody. In one embodiment of the antibodies or portions thereof described herein, the portion of the antibody comprises a Fc portion of the antibody. In one embodiment of the antibodies or portions thereof described herein, the portion of the antibody comprises a Fv portion of the antibody. In one embodiment of the antibodies or portions thereof described herein, the portion of the antibody comprises a variable domain of the antibody. In one embodiment of the antibodies or portions thereof described herein, the portion of the antibody comprises one or more CDR domains of the antibody.

Antibodies are prepared according to conventional methodology. Monoclonal antibodies may be generated using the method of Kohler and Milstein (Nature, 256:495, 1975). To prepare monoclonal antibodies useful in the invention, a mouse or other appropriate host animal is immunized at suitable intervals (e.g., twice-weekly, weekly, twice-monthly or monthly) with antigenic forms of OX1R. The animal may be administered a final “boost” of antigen within one week of sacrifice. It is often desirable to use an immunologic adjuvant during immunization. Suitable immunologic adjuvants include Freund's complete adjuvant, Freund's incomplete adjuvant, alum, Ribi adjuvant, Hunter's Titermax, saponin adjuvants such as QS21 or Quil A, or CpG-containing immunostimulatory oligonucleotides. Other suitable adjuvants are well-known in the field. The animals may be immunized by subcutaneous, intraperitoneal, intramuscular, intravenous, intranasal or other routes. A given animal may be immunized with multiple forms of the antigen by multiple routes. Briefly, the recombinant OX1R may be provided by expression with recombinant cell lines. In particular, OX1R may be provided in the form of human cells expressing OX1R at their surface. Following the immunization regimen, lymphocytes are isolated from the spleen, lymph node or other organ of the animal and fused with a suitable myeloma cell line using an agent such as polyethylene glycol to form a hydridoma. Following fusion, cells are placed in media permissive for growth of hybridomas but not the fusion partners using standard methods, as described (Coding, Monoclonal Antibodies: Principles and Practice: Production and Application of Monoclonal Antibodies in Cell Biology, Biochemistry and Immunology, 3rd edition, Academic Press, New York, 1996). Following culture of the hybridomas, cell supernatants are analyzed for the presence of antibodies of the desired specificity, i.e., that selectively bind the antigen. Suitable analytical techniques include ELISA, flow cytometry, immunoprecipitation, and western blotting. Other screening techniques are well-known in the field. Preferred techniques are those that confirm binding of antibodies to conformationally intact, natively folded antigen, such as non-denaturing ELISA, flow cytometry, and immunoprecipitation.

Significantly, as is well-known in the art, only a small portion of an antibody molecule, the paratope, is involved in the binding of the antibody to its epitope (see, in general, Clark, W. R. (1986) The Experimental Foundations of Modern Immunology Wiley & Sons, Inc., New York; Roitt, I. (1991) Essential Immunology, 7th Ed., Blackwell Scientific Publications, Oxford). The Fc′ and Fc regions, for example, are effectors of the complement cascade but are not involved in antigen binding. An antibody from which the pFc′ region has been enzymatically cleaved, or which has been produced without the pFc′ region, designated an F(ab′)2 fragment, retains both of the antigen binding sites of an intact antibody. Similarly, an antibody from which the Fc region has been enzymatically cleaved, or which has been produced without the Fc region, designated an Fab fragment, retains one of the antigen binding sites of an intact antibody molecule. Proceeding further, Fab fragments consist of a covalently bound antibody light chain and a portion of the antibody heavy chain denoted Fd. The Fd fragments are the major determinant of antibody specificity (a single Fd fragment may be associated with up to ten different light chains without altering antibody specificity) and Fd fragments retain epitope-binding ability in isolation.

Within the antigen-binding portion of an antibody, as is well-known in the art, there are complementarity determining regions (CDRs), which directly interact with the epitope of the antigen, and framework regions (FRs), which maintain the tertiary structure of the paratope (see, in general, Clark, 1986; Roitt, 1991). In both the heavy chain Fd fragment and the light chain of IgG immunoglobulins, there are four framework regions (FR1 through FR4) separated respectively by three complementarity determining regions (CDR1 through CDRS). The CDRs, and in particular the CDRS regions, and more particularly the heavy chain CDRS, are largely responsible for antibody specificity.

It is now well-established in the art that the non CDR regions of a mammalian antibody may be replaced with similar regions of conspecific or heterospecific antibodies while retaining the epitopic specificity of the original antibody. This is most clearly manifested in the development and use of “humanized” antibodies in which non-human CDRs are covalently joined to human FR and/or Fc/pFc′ regions to produce a functional antibody.

This invention provides in certain embodiments compositions and methods that include humanized forms of antibodies. As used herein, “humanized” describes antibodies wherein some, most or all of the amino acids outside the CDR regions are replaced with corresponding amino acids derived from human immunoglobulin molecules. Methods of humanization include, but are not limited to, those described in U.S. Pat. Nos. 4,816,567; 5,225,539; 5,585,089; 5,693,761; 5,693,762; and 5,859,205, which are hereby incorporated by reference. The above U.S. Pat. Nos. 5,585,089 and 5,693,761, and WO 90/07861 also propose four possible criteria which may used in designing the humanized antibodies. The first proposal was that for an acceptor, use a framework from a particular human immunoglobulin that is unusually homologous to the donor immunoglobulin to be humanized, or use a consensus framework from many human antibodies. The second proposal was that if an amino acid in the framework of the human immunoglobulin is unusual and the donor amino acid at that position is typical for human sequences, then the donor amino acid rather than the acceptor may be selected. The third proposal was that in the positions immediately adjacent to the 3 CDRs in the humanized immunoglobulin chain, the donor amino acid rather than the acceptor amino acid may be selected. The fourth proposal was to use the donor amino acid reside at the framework positions at which the amino acid is predicted to have a side chain atom within 3 A of the CDRs in a three dimensional model of the antibody and is predicted to be capable of interacting with the CDRs. The above methods are merely illustrative of some of the methods that one skilled in the art could employ to make humanized antibodies. One of ordinary skill in the art will be familiar with other methods for antibody humanization.

In one embodiment of the humanized forms of the antibodies, some, most or all of the amino acids outside the CDR regions have been replaced with amino acids from human immunoglobulin molecules but where some, most or all amino acids within one or more CDR regions are unchanged. Small additions, deletions, insertions, substitutions or modifications of amino acids are permissible as long as they would not abrogate the ability of the antibody to bind a given antigen. Suitable human immunoglobulin molecules would include IgG1, IgG2, IgG3, IgG4, IgA and IgM molecules. A “humanized” antibody retains a similar antigenic specificity as the original antibody. However, using certain methods of humanization, the affinity and/or specificity of binding of the antibody may be increased using methods of “directed evolution”, as described by Wu et al., /. Mol. Biol. 294:151, 1999, the contents of which are incorporated herein by reference.

Fully human monoclonal antibodies also can be prepared by immunizing mice transgenic for large portions of human immunoglobulin heavy and light chain loci. See, e.g., U.S. Pat. Nos. 5,591,669, 5,598,369, 5,545,806, 5,545,807, 6,150,584, and references cited therein, the contents of which are incorporated herein by reference. These animals have been genetically modified such that there is a functional deletion in the production of endogenous (e.g., murine) antibodies. The animals are further modified to contain all or a portion of the human germ-line immunoglobulin gene locus such that immunization of these animals will result in the production of fully human antibodies to the antigen of interest. Following immunization of these mice (e.g., XenoMouse (Abgenix), HuMAb mice (Medarex/GenPharm)), monoclonal antibodies can be prepared according to standard hybridoma technology. These monoclonal antibodies will have human immunoglobulin amino acid sequences and therefore will not provoke human anti-mouse antibody (KAMA) responses when administered to humans.

In vitro methods also exist for producing human antibodies. These include phage display technology (U.S. Pat. Nos. 5,565,332 and 5,573,905) and in vitro stimulation of human B cells (U.S. Pat. Nos. 5,229,275 and 5,567,610). The contents of these patents are incorporated herein by reference.

Thus, as will be apparent to one of ordinary skill in the art, the present invention also provides for F(ab′) 2 Fab, Fv and Fd fragments; chimeric antibodies in which the Fc and/or FR and/or CDR1 and/or CDR2 and/or light chain CDR3 regions have been replaced by homologous human or non-human sequences; chimeric F(ab′)2 fragment antibodies in which the FR and/or CDR1 and/or CDR2 and/or light chain CDR3 regions have been replaced by homologous human or non-human sequences; chimeric Fab fragment antibodies in which the FR and/or CDR1 and/or CDR2 and/or light chain CDR3 regions have been replaced by homologous human or non-human sequences; and chimeric Fd fragment antibodies in which the FR and/or CDR1 and/or CDR2 regions have been replaced by homologous human or non-human sequences. The present invention also includes so-called single chain antibodies.

The various antibody molecules and fragments may derive from any of the commonly known immunoglobulin classes, including but not limited to IgA, secretory IgA, IgE, IgG and IgM. IgG subclasses are also well known to those in the art and include but are not limited to human IgG1, IgG2, IgG3 and IgG4.

In another embodiment, the antibody according to the invention is a single domain antibody. The term “single domain antibody” (sdAb) or “VHH” refers to the single heavy chain variable domain of antibodies of the type that can be found in Camelid mammals which are naturally devoid of light chains. Such VHH are also called “Nanobody®”. According to the invention, sdAb can particularly be llama sdAb.

In one embodiment of the agents described herein, the agent is a polypeptide. In a particular embodiment the polypeptide is Orexin-A or Orexin-B; in other embodiments, the polypeptide is not Orexin-A or Orexin-B, i.e. the sequence of the polypeptide is not (is other than) SEQ ID NO: 2 or SEQ ID NO: 3. The Orexin-A and Orexin-B are not secreted by the body and are not naturally occurring but are synthetic, e.g. obtained by chemical synthesis, and are provided to the patient exogenously, e.g. as a bolus or particular dose. Functional equivalents of Orexin-A and Orexin-B are generally synthetic molecules which retain the function of Orexin-A and Orexin-B, e.g. they bind to the OX1R receptor and act as agonists of the receptor, and binding causes death of the cell in which the receptor to which they are bound is located. However, the functional equivalents differ from Orexin-A and Orexin-B in one or both of chemical composition and chemical (molecular) structure.

As used herein the term “orexin-A” has its general meaning in the art and refers to the amino acid sequence as shown by SEQ ID NO:2.

Orexin-A (SEQ ID NO: 2):
ₚeplpdccrqktcscrlyell
(where “ₚe” stands for “pyroglutamic acid”).

As used herein the term “orexin-B” has its general meaning in the art and refers to the amino acid sequence as shown by SEQ ID NO:3.

Orexin-B (SEQ ID NO: 3):
rsgppglqgr lqrllqasgn haagiltm

As used herein, a “functional equivalent of orexin” is a polypeptide which is capable of binding to OX1R, thereby promoting an OX1R activity according to the invention. The term “functional equivalent” includes fragments, mutants, and muteins of Orexin-A and Orexin-B. The term “functionally equivalent” thus includes any equivalent of orexins (i.e. Orexin-A or Orexin-B) obtained by altering the amino acid sequence, for example by one or more amino acid deletions, substitutions or additions such that the protein analogue retains the ability to bind to OX1R and promote an OX1R activity according to the invention (e.g. apoptosis of the cancer cell). Amino acid substitutions may be made, for example, by point mutation of the DNA encoding the amino acid sequence.

In some embodiments, the functional equivalent is at least about 80% homologous/identical to the corresponding protein. In a preferred embodiment, the functional equivalent is at least about 90% homologous/identical (e.g. at least about 90, 91, 92, 93, 94, 95, 96, 97, 98, or 99%) as assessed by any conventional analysis algorithm such as for example, the Pileup sequence analysis software (Program Manual for the Wisconsin Package, 1996). In these and other embodiments, the differences in identity between the amino acid sequence of a (modified) polypeptide agonist and the corresponding sequence (e.g. native Orexin-A or Orexin-B, i.e. SEQ ID NO: 2 or SEQ ID NO: 3) are due to the presence of one or more of: at least one substitution, at least one insertion, at least one deletion, and/or at least one amino acid modification, that is/are not present in the native sequence of Orexin-A or Orexin-B. In other words, the percentage of change is measured made relative to the native amino acid sequence of Orexin-A or Orexin-B, and the changes or modifications are not present in native Orexin-A or Orexin-B. The term “a functionally equivalent fragment” as used herein also may mean any fragment or assembly of fragments of Orexin that binds to OX1R and promote the OX1R activity according to the invention. Accordingly the present invention provides a polypeptide which comprises consecutive amino acids having a sequence which corresponds to the sequence of at least a portion of Orexin-A or Orexin-B, which portion binds to OX1R and promotes the OX1R activity according to the invention.

Functionally equivalent fragments may belong to the same protein family as the human Orexins identified herein. By “protein family” is meant a group of proteins that share a common function and exhibit common sequence homology. Homologous proteins may be derived from non-human species. In particular, the homology between functionally equivalent protein sequences is at least 25% across the whole of amino acid sequence of the complete protein. More In particular, the homology is at least 50%, even more In particular 75% across the whole of amino acid sequence of the protein or protein fragment. More In particular, homology is greater than 80% across the whole of the sequence. More In particular, homology is greater than 90% across the whole of the sequence. More In particular, homology is greater than 95% across the whole of the sequence.

In some embodiments, the last residue of SEQ ID NO:2, i.e. the methionine residue at position 28, is amidated. As used herein, the term “amidation,” has its general meaning in the art and refers to the process consisting of producing an amide moiety.

The polypeptides of the invention may be produced by any suitable means, as will be apparent to those of skill in the art. In order to produce sufficient amounts of polypeptides or functional equivalents thereof for use in accordance with the present invention, expression may conveniently be achieved by culturing under appropriate conditions recombinant host cells containing the polypeptide of the invention. In particular, the polypeptide is produced by recombinant means, by expression from an encoding nucleic acid molecule. Systems for cloning and expression of a polypeptide in a variety of different host cells are well known. When expressed in recombinant form, the polypeptide is in particular generated by expression from an encoding nucleic acid in a host cell. Any host cell may be used, depending upon the individual requirements of a particular system. Suitable host cells include bacteria mammalian cells, plant cells, yeast and baculovirus systems. Mammalian cell lines available in the art for expression of a heterologous polypeptide include Chinese hamster ovary cells, HeLa cells, baby hamster kidney cells and many others. Bacteria are also preferred hosts for the production of recombinant protein, due to the ease with which bacteria may be manipulated and grown. A common, preferred bacterial host is E coli.

Methods for producing amidated polypeptide are well known in the art and typically involve use of amidation enzyme. As used herein, the term “amidation enzyme” is defined as the enzymes which can convert the carboxyl group of a polypeptide to an amide group. Enzymes capable of C-terminal amidation of peptides have been known for a long time (Eipper et al. Mol. Endocrinol. 1987 November; 1 (11): 777). Examples of amidating enzymes include peptidylglycine α-monooxygenase (EC 1.14.17.3), herein referred to as PAM, and peptidylamidoglycolate lyase (EC 4.3.2.5), herein referred to as PGL. The preparation and purification of such PAM enzymes is familiar to the skilled worker and has been described in detail (M. Nogudi et al. Prot. Expr. Purif. 2003, 28: 293). An alternative to the “in vitro” amidation by means of PAM emerges when the enzyme is coexpressed in the same host cell with the precursor protein to be amidated (i.e the fusion protein of the present invention). This is achieved by introducing a gene sequence which codes for a PAM activity into the host cell under the control of a host-specific regulatory sequence. This expression sequence can either be incorporated stably into the respective chromosomal DNA sequence, or be present on a second plasmid parallel to the expression plasmid for the target protein (i.e. fusion protein of the present invention), or be integrated as second expression cassette on the same vector, or be cloned in a polycistronic expression approach in phase with the gene sequence which encodes the target protein (i.e. fusion protein of the present invention) under the control of the same promoter sequence. A further method for amidation is based on the use of protein-specific self-cleavage mechanisms (Cottingham et al. Nature Biotech. Vol. 19, 974-977, 2001). The amidation processes described above start from a C terminus of the target peptide which is extended by at least one amino acid glycine or alternatively interim peptide. Alternative methods, are also described in WO2007036299. Accordingly, in some embodiments, the nucleic acid sequence encoding for the orexin polypeptide is chosen to allow the amidation of said orexin polypeptide and thus may comprise additional codons that will code for a glycine-extended precursor. Typically, the glycine-extended precursor resembles YGXX, where Y represents the amino acid that shall be amidated and X represents any amino acid so that the amidation enzyme (e.g. PAM) catalyzes the production of the amidated polypeptide from said glycine-extended precursor. In some embodiments, the glycine-extended precursor is MGRR. In some embodiments, the nucleic acid sequence encoding for the orexin polypeptide that will allow amidation is SEQ ID NO:10.

SEQ ID NO: 10
gctccggcccccccggtcttcaaggccggcttcagcgcctgctgcaa
gcctcaggcaaccatgcagctgggatcctcacaatgggacgacgt

In some embodiments, the polypeptide of the invention is an immunoadhesin.

As used herein, the term “immunoadhesin” designates antibody-like molecules which combine the binding specificity of a heterologous protein (an “adhesin” which is able to bind to OX1R) with the effector functions of immunoglobulin constant domains. Structurally, the immunoadhesins comprise a fusion of an amino acid sequence with the desired binding specificity to OX1R (i.e., is “heterologous”), and an immunoglobulin constant domain sequence. The adhesin part of an immunoadhesin molecule typically is a contiguous amino acid sequence comprising at least the binding site for OX1R. In one embodiment, the adhesin comprises the polypeptides characterized by SEQ ID NO:2 or SEQ ID NO:3. The immunoglobulin constant domain sequence in the immunoadhesin may be obtained from any immunoglobulin, such as IgG-1, IgG-2, IgG-3, or IgG-4 subtypes, IgA (including IgA-1 and IgA-2), IgE, IgD or IgM.

The immunoglobulin sequence typically, but not necessarily, is an immunoglobulin constant domain (Fc region). Immunoadhesins can possess many of the valuable chemical and biological properties of human antibodies. Since immunoadhesins can be constructed from a human protein sequence with a desired specificity linked to an appropriate human immunoglobulin hinge and constant domain (Fc) sequence, the binding specificity of interest can be achieved using entirely human components. Such immunoadhesins are minimally immunogenic to the patient, and are safe for chronic or repeated use.

In one embodiment, the Fc region is a native sequence Fc region. In one embodiment, the Fc region is a variant Fc region. In still another embodiment, the Fc region is a functional Fc region. As used herein, the term “Fc region” is used to define a C-terminal region of an immunoglobulin heavy chain, including native sequence Fc regions and variant Fc regions. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The adhesion portion and the immunoglobulin sequence portion of the immunoadhesin may be linked by a minimal linker. The immunoglobulin sequence typically, but not necessarily, is an immunoglobulin constant domain. The immunoglobulin moiety in the chimeras of the present invention may be obtained from IgG1, IgG2, IgG3 or IgG4 subtypes, IgA, IgE, IgD or IgM, but typically IgG1 or IgG3.

The polypeptides of the invention, fragments thereof and fusion proteins (e.g. immunoadhesin) according to the invention can exhibit post-translational modifications, including, but not limited to glycosylations, (e.g., N-linked or O-linked glycosylations), myristylations, palmitylations, acetylations and phosphorylations (e.g., serine/threonine or tyrosine).

In specific embodiments, it is contemplated that polypeptides used in the therapeutic methods of the present invention may be modified in order to improve their therapeutic efficacy. Such modification of therapeutic compounds may be used to decrease toxicity, increase circulatory time, or modify biodistribution. For example, the toxicity of potentially important therapeutic compounds can be decreased significantly by combination with a variety of drug carrier vehicles that modify biodistribution. In example adding dipeptides can improve the penetration of a circulating agent in the eye through the blood retinal barrier by using endogenous transporters.

A strategy for improving drug viability is the utilization of water-soluble polymers. Various water-soluble polymers have been shown to modify biodistribution, improve the mode of cellular uptake, change the permeability through physiological barriers; and modify the rate of clearance from the body. To achieve either a targeting or sustained-release effect, water-soluble polymers have been synthesized that contain drug moieties as terminal groups, as part of the backbone, or as pendent groups on the polymer chain.

Polyethylene glycol (PEG) has been widely used as a drug carrier, given its high degree of biocompatibility and ease of modification. Attachment to various drugs, proteins, and liposomes has been shown to improve residence time and decrease toxicity. PEG can be coupled to active agents through the hydroxyl groups at the ends of the chain and via other chemical methods; however, PEG itself is limited to at most two active agents per molecule. In a different approach, copolymers of PEG and amino acids were explored as novel biomaterials which would retain the biocompatibility properties of PEG, but which would have the added advantage of numerous attachment points per molecule (providing greater drug loading), and which could be synthetically designed to suit a variety of applications.

Those of skill in the art are aware of PEGylation techniques for the effective modification of drugs. For example, drug delivery polymers that consist of alternating polymers of PEG and tri-functional monomers such as lysine have been used by VectraMed (Plainsboro, N.J.). The PEG chains (typically 2000 daltons or less) are linked to the a- and e-amino groups of lysine through stable urethane linkages. Such copolymers retain the desirable properties of PEG, while providing reactive pendent groups (the carboxylic acid groups of lysine) at strictly controlled and predetermined intervals along the polymer chain. The reactive pendent groups can be used for derivatization, cross-linking, or conjugation with other molecules. These polymers are useful in producing stable, long-circulating pro-drugs by varying the molecular weight of the polymer, the molecular weight of the PEG segments, and the cleavable linkage between the drug and the polymer. The molecular weight of the PEG segments affects the spacing of the drug/linking group complex and the amount of drug per molecular weight of conjugate (smaller PEG segments provides greater drug loading). In general, increasing the overall molecular weight of the block co-polymer conjugate will increase the circulatory half-life of the conjugate. Nevertheless, the conjugate must either be readily degradable or have a molecular weight below the threshold-limiting glomular filtration (e.g., less than 60 kDa).

In addition, to the polymer backbone being important in maintaining circulatory half-life, and biodistribution, linkers may be used to maintain the therapeutic agent in a pro-drug form until released from the backbone polymer by a specific trigger, typically enzyme activity in the targeted tissue. For example, this type of tissue activated drug delivery is particularly useful where delivery to a specific site of biodistribution is required and the therapeutic agent is released at or near the site of pathology. Linking group libraries for use in activated drug delivery are known to those of skill in the art and may be based on enzyme kinetics, prevalence of active enzyme, and cleavage specificity of the selected disease-specific enzymes. Such linkers may be used in modifying the protein or fragment of the protein described herein for therapeutic delivery.

In one embodiment, the OX1R agonist is an aptamer. Aptamers are a class of molecule that represents an alternative to antibodies in term of molecular recognition. Aptamers are oligonucleotide or oligopeptide sequences with the capacity to recognize virtually any class of target molecules with high affinity and specificity. Such ligands may be isolated through Systematic Evolution of Ligands by EXponential enrichment (SELEX) of a random sequence library. The random sequence library is obtainable by combinatorial chemical synthesis of DNA. In this library, each member is a linear oligomer, eventually chemically modified, of a unique sequence. Peptide aptamers consists of a conformationally constrained antibody variable region displayed by a platform protein, such as E. coli Thioredoxin A that are selected from combinatorial libraries by two hybrid methods.

The term “pancreatic cancer” or “pancreas cancer” as used herein relates to cancer which is derived from pancreatic cells. In particular, pancreatic cancer included pancreatic adenocarcinoma (e.g., pancreatic ductal adenocarcinoma) as well as other tumors of the exocrine pancreas (e.g., serous cystadenomas), acinar cell cancers, intraductal papillary mucinous neoplasms (IPMN) and pancreatic neuroendocrine tumors (such as insulinomas). The cancer may be metastatic cancer. The cancer cells and or tumors that are treated may or may not be resistant to conventional cancer therapy, i.e. the cells in a tumor may exhibit either primary or acquired resistance to conventional cancer therapy and yet they are responsive to (killed by) administration or one or more OX1R agonists.

In some embodiments, the OX1R agonist of the invention is administered to the subject with a therapeutically effective amount. In some aspects, the subject is identified or classified as having pancreatic cancer. The methods disclosed herein may comprise a step of identifying such subjects.

By a “therapeutically effective amount” is meant a sufficient amount of OX1R to treat pancreatic cancer at a reasonable benefit/risk ratio applicable to any medical treatment. It will be understood that the total daily usage of the compounds and compositions of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidental with the specific polypeptide employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. In particular, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, in particular from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day. In some aspects, the agonist is administered at a rate of about 0.01, 0.1, 1 or 10 μmol/kg of body weight per day. Administration typically involves delivery of a bolus of agonist by one or more of the indicated means, so that, for example, in the case of the agonists orexin A and/or B, the concentration of the agonist within the patient's body is greater than that which occurs in nature, e.g. is greater than a normal physiological level. A “bolus” refers to administration of a discrete amount of medication, drug or other compound in order to raise its concentration in blood or plasma to a desired and effective level. For example, after administration, the concentration of agonist in plasma is generally at least about 60 pg/ml and usually greater, e.g. about 70 pg/ml or higher, e.g. greater than about 100, or 1000 pg/ml.

The OX1R agonist of the invention may be combined with pharmaceutically acceptable excipients, and optionally sustained-release matrices, such as biodegradable polymers, to form therapeutic compositions.

“Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

In the pharmaceutical compositions of the present invention for oral, sublingual, subcutaneous, intramuscular, intravenous, transdermal, local (e.g. intratumoral) or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal, and intranasal administration forms and rectal administration forms.

In particular, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi.

Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The OX1R agonist of the invention can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifuCASK agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active polypeptides in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject.

The OX1R agonist of the invention may be formulated within a therapeutic mixture to comprise about 0.0001 to 1.0 milligrams, or about 0.001 to 0.1 milligrams, or about 0.1 to 1.0 or even about 10 milligrams per dose or so. Multiple doses can also be administered.

In addition to the compounds of the invention formulated for parenteral administration, such as intravenous or intramuscular injection, other pharmaceutically acceptable forms include, e.g. tablets or other solids for oral administration; liposomal formulations; time release capsules; and any other form currently used.

In some embodiments, the OX1R agonist of the invention is used in combination with a chemotherapeutic agent. Chemotherapeutic agents include, but are not limited to alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall; dynemicin, including dynemicin A; bisphosphonates, such as clodronate; an esperamicin; as well as neocarzinostatin chromophore and related chromoprotein enediyne antiobiotic chromophores, aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxy doxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and doxetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-1 1); topoisomerase inhibitor RFS 2000; difluoromethylomithine (DMFO); retinoids such as retinoic acid; capecitabine; and pharmaceutically acceptable salts, acids or derivatives of any of the above.

A further object of the invention relates to a method for treating a pancreatic cancer in a subject in thereof comprising the steps consisting of i) determining the expression level of OX1R in a tumour tissue sample obtained from the subject, ii) comparing the expression level determined at step i) with a reference value and iii) administering the subject with a therapeutically effective amount of an OX1R agonist when the level determined at step i) is higher than the reference value.

The expression level of OX1R may be determined by any well known method in the art. For example methods for determining the quantity of mRNA are well known in the art. Typically the nucleic acid contained in the samples (e.g., cell or tissue prepared from the patient) is first extracted according to standard methods, for example using lytic enzymes or chemical solutions or extracted by nucleic-acid-binding resins following the manufacturer's instructions. The extracted mRNA is then detected by hybridization (e. g., Northern blot analysis) and/or amplification (e.g., RT-PCR). Preferably quantitative or semi-quantitative RT-PCR is preferred. Real-time quantitative or semi-quantitative RT-PCR is particularly advantageous. Alternatively an immunohistochemistry (IHC) method may be used. IHC specifically provides a method of detecting targets in a sample or tissue specimen in situ. The overall cellular integrity of the sample is maintained in IHC, thus allowing detection of both the presence and location of the targets of interest (i.e. OX1R). Typically a sample is fixed with formalin, embedded in paraffin and cut into sections for staining and subsequent inspection by light microscopy. Current methods of IHC use either direct labeling or secondary antibody-based or hapten-based labeling. Examples of known IHC systems include, for example, EnVision™ (DakoCytomation), Powervision® (Immunovision, Springdale, Ariz.), the NBA™ kit (Zymed Laboratories Inc., South San Francisco, Calif.), HistoFine® (Nichirei Corp, Tokyo, Japan). In particular embodiment, a tumor tissue section may be mounted on a slide or other support after incubation with antibodies directed against OX1R. Then, microscopic inspections in the sample mounted on a suitable solid support may be performed. For the production of photomicrographs, sections comprising samples may be mounted on a glass slide or other planar support, to highlight by selective staining the presence of the proteins of interest.

A “reference value” can be a “threshold value” or a “cut-off value”. Typically, a “threshold value” or “cut-off value” can be determined experimentally, empirically, or theoretically. A threshold value can also be arbitrarily selected based upon the existing experimental and/or clinical conditions, as would be recognized by a person of ordinary skilled in the art. The threshold value has to be determined in order to obtain the optimal sensitivity and specificity according to the function of the test and the benefit/risk balance (clinical consequences of false positive and false negative). Typically, the optimal sensitivity and specificity (and so the threshold value) can be deteiinined using a Receiver Operating Characteristic (ROC) curve based on experimental data. Typically, the threshold value is derived from the OX1R expression level (or ratio, or score) determined in a tumour tissue sample derived from one or more subjects having sufficient amount of OX1R level to get an efficient treatment with the OX1R agonist. Furthermore, retrospective measurement of the OX1R expression levels (or ratio, or scores) in properly banked historical subject samples may be used in establishing these threshold values.

A further object of the invention relates to methods of decreasing the size of an established pancreatic cancer tumor in a patient in need thereof. The methods comprise a step of administering to the patient a therapeutically effective amount of an OX1R agonist, the amount being sufficient to decrease the size of the tumor. The methods may also include a step of identifying a patient suffering from or harboring a pancreatic tumor, e.g. an established, detectable tumor. An “established” tumor is a tumor with a size sufficient to be detected using usual detection methods, e.g. usually the tumor is of a size of at least about 0.5 cm or greater in at least one dimension and is detectable e.g. by palpation, by imaging (e.g. X-ray, positron emission tomography—computed tomography (PET/CT), magnetic resonance imaging (MRI), ultrasound, etc.), or by some other means. 1-, 2- and/or 3-dimensional measurements may be used to determine tumor size and/or volume). Administration of at least one OX1R agonist results in a decrease in tumor size of, e.g. at least about 10%, and usually about 20, 30, 40, 50, 60, 70, 80, 90 or 100%, compared to the size of an equivalent (e.g. control) untreated tumor. A 100% decrease indicates complete eradication of detectable tumor.

A further object of the invention relates to methods of preventing or slowing pancreatic tumor growth in a patient in need thereof, comprising administering to said patient a therapeutically effective amount of an OX1R agonist, the amount being sufficient to prevent or slow the growth of at least one pancreatic tumor. The rate of tumor growth (increase in size) is slowed, for example, at least by about 10%, and usually by about 20, 30, 40, 50, 60, 70, 80, 90 or 100%, compared to the rate of growth of a comparable (e.g. control) untreated tumor. A 100% decrease in tumor growth means that the tumor stops growing (tumor growth in halted), and the tumor may even decrease in size (negative growth rate) in response to administration of the agonist.

A further object of the invention relates to a method for screening a drug for the treatment of pancreatic cancer comprising the steps of i) providing a plurality of test substances ii) determining whether the test substances are OX1R agonists and iii) positively selecting the test substances that are OX1R agonists.

Typically, the screening method of the invention involves providing appropriate cells which express the orexin-1 receptor on their surface. Such cells include cells from mammals, yeast, Drosophila or E. coli. In particular, a polynucleotide encoding the orexin-1 receptor is used to transfect cells to express the receptor. The expressed receptor is then contacted with a test substance and an orexin-1 receptor ligand (e.g. orexins), as appropriate, to observe activation of a functional response such as recruitment of SHP-2 and induction of cell apoptosis of the cell. Functional assays may be performed as described in El Firar A, Voisin T, Rouyer-Fessard C, Ostuni M A, Couvineau A, Laburthe M. Discovery of a functional immunoreceptor tyrosine-based switch motif in a 7-transmembrane-spanning receptor: role in the orexin receptor OX1R-driven apoptosis. FASEB J. 2009 December; 23(12):4069-80. doi: 10.1096/fj.09-131367. Epub 2009 Aug. 6. In particular comparison steps may involve to compare the activity induced by the test substance and the activity induce by a well known OX1R agonist such as orexin. In particular substances capable of having an activity similar or even better than a well known OX1R agonist are positively selected.

Typically, the screening method of the invention may also involve screening for test substances capable of binding of to orexin-1 receptor present at cell surface. Typically the test substance is labelled (e.g. with a radioactive label) and the binding is compared to a well known OX1R agonist such as orexin. The preparation is incubated with labelled OX1R and complexes of test substances bound to NGAL are isolated and characterized according to routine methods known in the art. Alternatively, the OX1R may be bound to a solid support so that binding molecules solubilized from cells are bound to the column and then eluted and characterized according to routine methods. In another embodiment, a cellular compartment may be prepared from a cell that expresses a molecule that binds NGAL such as a molecule of a signalling or regulatory pathway modulated by NGAL. The preparation is incubated with labelled NGAL in the absence or the presence of a candidate compound. The ability of the candidate compound to bind the binding molecule is reflected in decreased binding of the labelled ligand.

Typically, the candidate compound is selected from the group consisting of small organic molecules, peptides, polypeptides or oligonucleotides.

The test substances that have been positively selected may be subjected to further selection steps in view of further assaying its properties for the treatment of pancreatic cancer. For example, the candidate compounds that have been positively selected may be subjected to further selection steps in view of further assaying its properties on animal models for pancreatic cancer.

The above assays may be performed using high throughput screening techniques for identifying test substances for developing drugs that may be useful to the treatment of pancreatic cancer. High throughput screening techniques may be carried out using multi-well plates (e.g., 96-, 389-, or 1536-well plates), in order to carry out multiple assays using an automated robotic system. Thus, large libraries of test substances may be assayed in a highly efficient manner. More particularly, stably-transfected cells growing in wells of micro-titer plates (96 well or 384 well) can be adapted to high through-put screening of libraries of compounds. Compounds in the library will be applied one at a time in an automated fashion to the wells of the microtitre dishes containing the transgenic cells described above. Once the test substances which activate the apoptotic signals are identified, they can be positively selected for further characterization. These assays offer several advantages. The exposure of the test substance to a whole cell allows for the evaluation of its activity in the natural context in which the test substance may act. Because this assay can readily be performed in a microtitre plate format, the assays described can be performed by an automated robotic system, allowing for testing of large numbers of test samples within a reasonably short time frame. The assays of the invention can be used as a screen to assess the activity of a previously untested compound or extract, in which case a single concentration is tested and compared to controls. These assays can also be used to assess the relative potency of a compound by testing a range of concentrations, in a range of 100 μM to 1 μM, for example, and computing the concentration at which the apoptosis is maximal.

The invention will be further illustrated by the following figures and examples. However, these examples and figures should not be interpreted in any way as limiting the scope of the present invention.

### FIGURES

FIG. 1 shows tumoral reduction induced by orexin-A injection in nude mice xenografted with AsPC-1 cells.

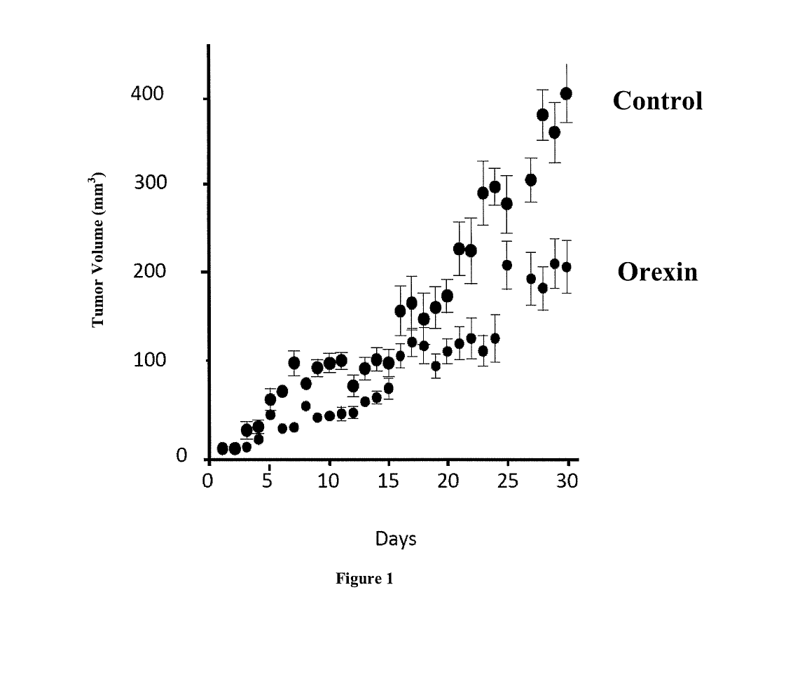

FIG. 2A-D. Immunohistochemical expression of the Orexin Receptor (OX1R) by tumors (A and B) and normal pancreas (C and D). OX1R is strongly expressed by tumor cells in PDAC, but is not detected in the surrounding stroma (A); at higher magnification, the staining is located to the membrane (arrows, B) and cytoplasm, and scored at 300 (intensity 3 on 100% of tumor cells, see “Materials and Methods”). OX1R was not detected in normal pancreas (C). At higher magnification (D), the normal duct was negative. Bar=200 μm for (A), 50 μm for (B), 120 μm for (C) and 40 μm for (D).

FIGS. 3A and B. Expression of OX1R in PDCA cells. A, RT-PCR analysis of OX1R (top panel) or OX2R (middle panel) mRNA from AsPC-1 cells, SW1990 cells, parental HPAF-II cell, HPAF-II cells expressing recombinant OX1R, CHO/OX1R cells, and CHO/OX2R cells. Controls are shown in the last lane (H₂O) in absence of DNA template. RT-PCR analysis of β-actin mRNA was used as control (bottom panel). B, shows the immunostaining of OX1R in paraformaldehyde-fixed and paraffin-embedded section from pellets of AsPC-1 cells (left panel) and HPAF-II cells (right panel) cultured in standard medium in the presence of FCS.

FIG. 4A-C. Effect of orexin-A on apoptosis in AsPC-1 cells. A, SHP-2 protein tyrosine phosphatase inhibitor, NSC-87877, blocks orexin-induced apoptosis. AsPC-1 cells were challenged with (black bars) or without (white bars) 1 μM orexin-A for 48 hr in the absence or presence of NSC-87877 (50 μM). Apoptosis was measured by determination of annexin V-PE binding, and results are expressed as the percentage of apoptotic cells; B and C: Indirect immunostaining of activated caspase-3 in AsPC-1 cells in the presence or absence of orexin-A. Paraformaldehyde-fixed AsPC-1 cells were challenged with (orexin-A) or without (basal) 1 μM orexin-A for 48 hr. Activated caspase-3 immunostaining is shown in B and scored in C. Results are means±SE of three separate experiments. ***p<0.001; ns, non-significant.

FIGS. 5A and B. Effect of orexin-A on apoptosis in OX1R expressing recombinant OX1R/HPAF-II cells. A, Parental HPAF-II and recombinant OX1R/HPAF-II cells were challenged with (black bars) or without (white bars) 1 μM orexin-A for 48 hr in the absence or presence of the SHP-2 protein tyrosine phosphatase inhibitor, NSC-87877 (50 μM). Apoptosis was measured by determination of annexin V-PE binding. Results are expressed as the percentage of apoptotic cells, and are the means±SE of three separate experiments. ***p<0.001; ns, non-significant; B, paraformaldehyde-fixed HPAF-II cells and recombinant OX1R/HPAF-II cells were challenged with or without (Basal) 1 μM orexin-A for 48 hr. Indirect immunostaining of activated caspase-3 in parental HPAF-II cells (left panels) and recombinant OX1R/HPAF-II cells (right panels) in the presence (bottom panels) or tabsence (top panels) of orexin-A is illustrated in B.

FIGS. 6A and B. Effect of daily inoculation of orexin-A on the growth of tumors developed by xenografting human PDCA cells in nude mice. AsPC-1 cells were inoculated in the flank of nude mice at day 0. Mice were injected daily intraperitoneally with 100 μl of orexin-A solutions starting at day 0 (◯) or day 14 (▴) or with 100 μl of PBS () for controls. A, the daily treatment corresponded to 1 μmoles of orexin-A/Kg. Inset represents the tumor weight measured at the end of the experiment after the mice were sacrificed; B, mice received 0.01, 0.1, 1 or 10 μmoles of orexin-A/kg. After 30 days of treatment, mice were sacrificed and tumor volume and weight were then recorded. The development of tumors was followed by caliper measurement. Data are the means±SE of 6 tumors in each group. *** p<0.01 versus control.

FIG. 7A-F. Indirect immunostaining of activated caspase-3 in xenografted AsPC-1 tumors resected from nude mice. Paraformaldehyde-fixed xenografted AsPC-1 tumors from nude mice treated daily (B, D and F) by intraperitoneal injections with 1 μmoles/Kg orexin-A or not (A, C and E). Orexin-A induced tumoral cell death (B), as detected by Hemalum Eosin Safran (HES) staining, which correlated with apoptosis induction assessed by strong immunostaining of activated caspase-3 after 30 days of orexin-A treatment; (F). OX1R immunostaining localisation was similar under control and orexin-A treatment conditions.

FIG. 8A-C. Effect of daily inoculation of orexin-A on the growth of tumors developed by xenografting OX1R expressing recombinant HPAF-II cells in nude mice. Parental HPAF-II (A) or recombinant OX1R/HPAF-II/cells (B) were inoculated in the flank of nude mice at day 0. Mice were injected daily intraperitoneally with 100 μl of 1 μmoles of orexin-A/Kg solutions starting at day 0 for both cell lines (◯) or day 28 for OX1R/HPAF-II cells (▴) or with 100 μl of PBS () for controls. The development of tumors was followed by caliper measurement; C. Formalin-fixed xenografted HPAF-II or OX1R/HPAF-II tumors from nude mice intraperitoneally injected daily or not with 1 μmoles/Kg orexin-A were analyzed by cleaved caspase-3 immunostaining. Cleaved caspase-3 positive cells were counted in 10 different fields, each comprising 500 tumoral cells, in the presence (black bars) or absence (white bars) of 50 days orexin-A treatment. Data are the means±SE of 6 tumors in each group; *** p<0.01 versus control.

FIG. 9. Effect of Orexin-B anti-OX1R antibodies on cell growth of AsPC-1 cells. Cells were incubated with 0.1 μM of OxB or antibodies for 48 h in culture medium and then cells were counted in order to estimate the cellular growth.

### EXAMPLES

### Example 1

Orexins are hypothalamic peptides involved in sleep/wake control. We have shown that orexins promote robust apoptosis in colorectal cancer cells. The cell death is mediated by the orexin 1 receptor (OX1R) through an original mechanism involving the presence of two ITIMs (immunoreceptor tyrosine inhibitory motif) in the OX1R sequence and the recruitment and activation of the tyrosine phosphatase SHP-2. OX1R, a class I GPCR, is aberrantly expressed in primary colorectal tumors and liver metastases. Pancreatic ductal adenocarcinomas (PAC) are highly malignant neoplasms with poor prognosis. Chemotherapy treatment shows a poor response rate. We have demonstrated the expression of OX1R in a large percentage of pancreatic adenocarcinomas by immunohistochemistry suggesting the ectopic OX1R expression in 98% of tested PAC.

The aims of this study were: 1/ to investigate the presence of OX1R in human PAC cell lines and to analyze orexin-A effects in relation to apoptosis; 2/ to develop an in vivo heterotopic xenograft model from the cell lines expressing OX1R, for the study of tumor growth in response to Orexin-A. The expression of OX1R was studied at mRNA (RT-PCR), proteins (immunocytochemistry) and functional levels in 3 PAC cell lines (AsPC-1, HPAF-II and SW1990). The development of an animal model (heterotopic xenograft) from the cell line expressing OX1R, has allowed studying the effect of Orexin-A in tumor growth. Resected tumors were analyzed by immunohistochemistry. Only AsPC-1 cell line expresses OX1R.

The treatment with Orexin-A promoted a 32% cell growth inhibition by promoting a mitochondrial apoptosis. Using the SHP inhibitor NSC-87877, we demonstrated the ability of the inhibitor to reverse orexin-induced apoptosis in AsPC-1 cells. Orexin-A injection in nude mice xenografted with AsPC-1 cells, has declined 49% of tumor progression in treated cases. All the tumors corresponded to poorly differentiated adenocarcinomas expressing cytokeratin 7, CA9 (hypoxia marker) and OX1R. Induction of apoptosis was observed in Orexin-A treated tumors (activated caspase-3).

In conclusion this work has demonstrated the antitumor and proapoptotic effects of orexins in PAC. In this context, orexin receptors represent a new promising target in pancreatic antineoplastic therapy and/or preclinical diagnostic.

### Example 2

### Orexin Receptor, OX1R, in Pancreatic Cancer: A New Proapoptotic Target

Objective Resistance to therapy is the main obstacle to a cure in pancreatic ductal adenocarcinoma cancer (PDAC), justifying the search for new therapeutical targets. The expression and role of the proapoptotic GPCR, OX1R Here, was investigated in a large series of human PDAC. Seventy patients with PDAC, treated with surgery, were analysed for OX1R expression by immunohistochemistry. PDAC cell lines were used to study the role of OX1R in cell apoptosis in vitro and tumor growth in xenografted mice in vivo.

### Materials and Methods

Patients and Tissue Collection

Seventy patients with PDAC, treated with surgery (pancreato-duodenectomy n=61; left pancreatectomy n=9; total pancreatectomy n=3) from April 1997 to December 2004 were selected from the files of the Department of Pathology at the Beaujon Hospital, Clichy, France. Charts from patients were retrospectively reviewed for clinical and pathological data. No patients received chemotherapy or radiation therapy preoperatively. The following data were recorded: age, gender, recurrence, disease-free survival (DFS) and overall survival (OS), tumor size, TNM stage, lymph node metastasis, differentiation. The studied population included 38 men and 35 women. The median age at surgery was 60 years (range 34-76). The tumor stage was T1 in 3 patients, T2 in 8 patients and T3 in 59 patients. The median tumor size was 30 mm (range 10-100 mm). Lymph node metastases were present in 52 patients. Tumors were well—(n=36), moderately—(n=22) or poorly—(n=12) differentiated. The median follow-up was 677 days (range 142-4294). Fifty-five patients (78.6%) died of the disease during the time of the study.

Tissue microarray (TMA) blocks were produced from representative paraffin blocks from the 70 PDAC surgical samples using a tissue arrayer (Manual Tissue Arrayer-MTA1, Beecher Instruments, WI, USA). For each tumor specimen, three 1 mm cores were randomly selected and included in the TMA blocks. A total of 3 TMA blocks were produced. The use of human material was approved by the Institutional Review Board (CEERB GHU Paris Nord N9RB12-059 and 12-033).

Cell Line Culture

The human pancreatic cancer cell lines were obtained from the American Type Culture Collection (Manassas, Va.). Cell lines were established from human metastasis of pancreatic ductal adenocarcinomas, i.e., splenic metastasis for SW 1990, and peritoneal ascites for AsPC-1 and HPAF-II. Cells were routinely cultured in 25 cm² plastic flasks (Costar), and maintained at 37° C. in a humidified atmosphere of 5% CO₂/air. SW 1990 cells were grown in Dulbecco's modified Eagle's medium (DMEM) containing 4.5 g glucose/L; AsPC-1 cells were grown in RPMI 1640 and HPAF-II in Minimum essential Medium (MEM). All cell lines were supplemented with 10% FCS, 100 μg/mL streptomycin and 100 units/mL penicillin (Invitrogen).

The HPAF-II/hOX1R cell line, expressing recombinant OX1 receptor, was obtained as previously described (Voisin T, El Firar A, Rouyer-Fessard C, et al. A hallmark of immunoreceptor, the tyrosine-based inhibitory motif ITIM, is present in the G protein-coupled receptor OX1R for orexins and drives apoptosis: a novel mechanism. FASEB J 2008; 22:1993-2002). Briefly, the human wild-type OX1R, cloned into the expression vector pEYFP in fusion with the yellow fluorescent protein (YFP) coding gene in the C-terminal position, was stably transfected in the parental HPAF-II cells, which do not express OX1R. HPAF-II/hOX1R cells were selected in the presence of geneticin (G418; 0.5 mg/ml), then cloned and cultured as the parental HPAF-II cells as described above.

RT-PCR Assays

For cultured cell lines (AsPC-1, SW 1990, HPAF-II and HPAF-II/OX1R) and control CHO cells expressing either recombinant OX1R (CHO/OX1R) or recombinant OX2R (CHO/OX2R), total RNA (RNA_{T}) was extracted from cells by using RNeasy® Mini Kit (Qiagen). Quality and integrity of RNA were evaluated using a Genequant RNA/DNA calculator (Pharma Biotech). All RNA_{T} were reverse-transcribed by using oligo (dT) primers. cDNA mixture was amplified by using human OX1R sense primer (5′-CCTGTGCCTCCAGACTATGA-3′; SEQ ID NO: 4); and OX1R antisense primer (5′-ACACTGCTGACATTCCATGA-3′ SEQ ID NO: 5); OX2R sense primer (5′-TAGTTCCTCAGCTGCCTATC-3′ SEQ ID NO: 6); and OX2R antisense primer (5′-CGTCCTCATGTGGTGGTTCT-3′ SEQ ID NO: 7); or β-actin sense primer (5′-ATCTGGCACCACACCTTCTACAATGAGCTGCG-3′ SEQ ID NO: 8); and β-actin antisense primer (5′-CGTCATACTCCTGCTTGCTGATCCACATCTGC-3′ SEQ ID NO: 9). PCR amplification was carried out using a Thermal cycler (Applied Biosystem 2720). Each of the 30 amplification cycles consisted of 95° C. for 30 seconds, 60° C. for 30 seconds, and 72° C. for 30 seconds. Amplicons were separated by electrophoresis in 1% agarose gel, stained with safe SYBR® Green (Invitrogen), and viewed under ultraviolet illumination.

Quantification of Apoptotic Cells by Annexin V Labeling

AsPC-1, SW 1990, HPAF-II and HPAF-II/hOX1R cells (seeded at 5×10⁴ cells/well) were grown in 24-well plates for 24 hr under the culture conditions described above. The culture medium was then replaced every 24 hr with fresh medium with or without orexin-A (GL Biochemicals) at a concentration of 1 μM in the presence or absence of the SHP-2 inhibitor, NSC-87877 (50 μM) (Calbiochem, VWR International SAS, France). At the end of the treatment (48 hr), apoptotic cells were determined using the Guava Nexin™ kit (Guava Technologies, Hayward, Calif., USA), which discriminates between apoptotic and non-apoptotic cells (Rouet-Benzineb P, Rouyer-Fessard C, Jarry A, et al. Orexins Acting at Native OX1 Receptor in Colon Cancer and Neuroblastoma Cells or at Recombinant OX1 Receptor Suppress Cell Growth by Inducing Apoptosis. J Biol Chem 2004; 279:45875-86), analyzed with the Guava Personal Cell Analysis (PCA) system (Merck-Millipore, Guyancourt, France), and counted (2,000 events) (Voisin et al, 2008, as above). Results are expressed as the percentage of apoptotic phycoerythrin-labelled Annexin V (Annexin V-PE) positive cells and are the means of 3 independent analyses.

Tumorigenicity Assay in Nude Mice

Exponentially growing AsPC-1, HPAF-II and HPAF-II/OX1R cells were harvested, washed with PBS and then resuspended in gelatin (2% solution type B from bovine skin, Sigma). Nude mice were anesthetized by intraperitoneal injection of a mixture containing 254 of Rompun 2% (Xylasine, Bayer) and 200 μL of Imalgene 500 (Ketamine 50 mg/mL, Merial) in 400 μL of PBS. Cells (10⁶/100 μL) were then inoculated subcutaneously into the flank of mice. All nude mice developed tumors at the site of inoculation between day 3 and 10. Tumor development was followed by caliper measurements in 2 dimensions (L and W), and the volume (V) of the tumor was calculated with the formula for a prolate ellipsoid (V=L×W²×π/6) as reported (Maoret J-J, Anini Y, Rouyer-Fessard C, et al. Neurotensin and a non-peptide neurotensin receptor antagonist control human colon cancer cell growth in cell culture and in cells xenografted into nude mice. Int J Cancer 1999; 80:448-54; Stragand J J, Barlogie B, White R A, et al. Biological Properties of the Human Colonic Adenocarcinoma Cell Line SW 620 Grown as a Xenograft in the Athymic Mouse. Cancer Res 1981; 41:3364-9). For treatment with orexin-A (GL Biochemicals), the peptide was dissolved in PBS, and 0.01, 0.1, 1 or 10 μmol/kg of body weight were administered by intraperitoneal injections. Control mice received PBS. No adverse effect of orexin-A could be observed during treatment. At the end of the in vivo experiments, mice were necropsied. The xenografted tumors were then resected, weighed (MARK electronic balance, Bel engineering) and analyzed. Paraffin-embedded tissues were cut in 3 μm sections, which were either stained with hematoxylin-eosin or used for immunohistochemistry.

Immunohistochemical Procedures

After dewaxing, rehydrating tumor paraffin sections, and antigen retrieval by pretreatment with high temperature at pH 9, immunohistochemical procedures were carried out using an automated immunohistochemical stainer according to the manufacturer's guidelines (Bond-Max slide stainer, Menarini, Leica Microsystems). For immunohistochemistry on cell lines, cells in pellets were fixed in formalin, embedded in cell blocks (Shandon Cytoblock; Thermo Scientific; USA) and cut into 3 μm sections. OX1R evaluation was performed in human PDAC, included in TMA, in xenografted tumors and in cell lines (AsPC-1, HPAF-II and HPAF-II/hOX1R). After antigen retrieval, 3 μm cells or tissue sections were incubated for 30 minutes with a polyclonal anti-OX1R antibody (My Bio Source; polyclonal goat; 1/300), rinsed, and then incubated with a biotinylated secondary rabbit anti-goat antibody (Vector BA-500; 1/400). Sections were rinsed and incubated with Streptavidin (TrekAvidin-HRP; Biocare Medical) and DAB ultraview detection kit (Bond Polymer Refine detection; DS9800; Leica Microsystems). Substitution of the primary antibody with PBS was used as a negative control. OX1R immunostaining was evaluated by two investigators (TV and AC) by calculating a score (0-300) obtained by multiplying the intensity (negative, 0; weak, 1; moderate, 2; and strong, 3) by the percentage of stained cells. The pattern of expression (cytoplasmic, membranous, and nuclear) was also recorded, and a mean score was calculated for each tumor. Internal positive controls consisted of normal pancreatic islets while the HEK/hOX1R cell line served as an external positive control. Specificity of the immunostaining was verified by incubation of OX1R antibody with its homologous immunogenic peptide or omission of the primary antibody. Apoptosis determination was performed in tumor cell lines (AsPC-1, HPAF-II and HPAF-II/hOX1R) and resected xenografts, treated or not with orexin-A. Three μm cell sections were immunolabelled for activated caspase-3 after antigen retrieval (ABGENT; cleaved caspase 3; polyclonal rabbit; 1/100) using a detection kit (Bond Polymer Refine detection; DS9800; Leica Microsystems). Substitution of the primary antibody with PBS was used as a negative control. External positive controls consisted of normal lymph nodes. Cleaved caspase-3 immunostaining was determined by calculating the percentage of tumor cells stained in 10 fields of xenografted tumor cells, each field comprising 500 tumor cells.

Statistical Analysis

Mann-Whitney non-parametric tests were utilized to compare categorical with continuous tumor variables where the number of categories was two. When the number of categories was greater than two, ANOVA (analysis of variance) tests were used instead. Data were analyzed with the GraphPad Prism 5.04 statistical software for Windows. All statistical tests were 2-sided. The critical level of statistical significance was set at p<0.05.

Results

Aberrant OX1R Expression in Human Pancreatic Adenocarcinomas

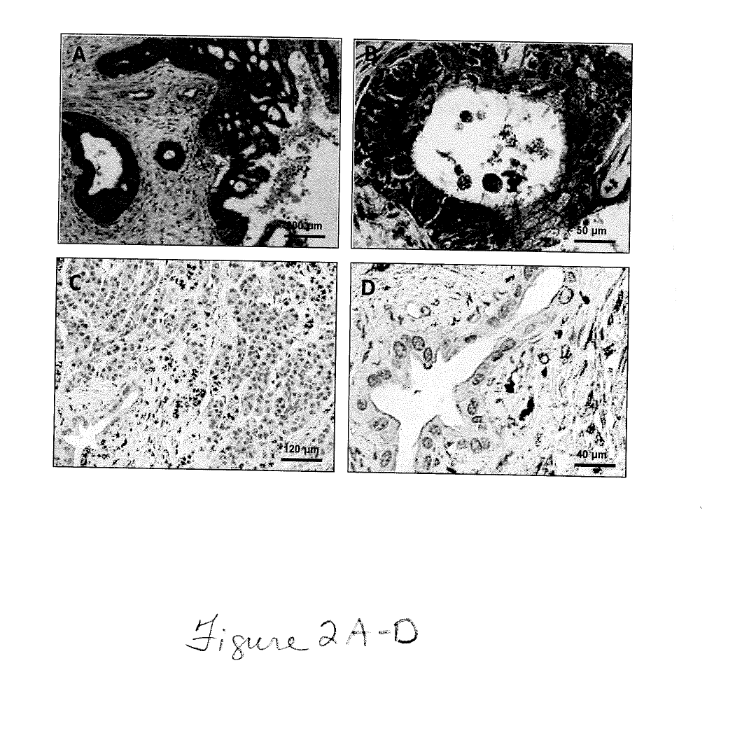

The expression of OX1R was determined by immunohistochemical (IHC) in 73 human PDAC versus normal pancreatic tissue. Seventy primary pancreatic tumors (70/73; 96%) expressed OX1R, as shown by positive immunoreactivity (FIGS. 2A & 2B). OX1R expression was mainly observed in the cytoplasm and membranes. Scores ranging from 0 to 300 based on OX1R immunoreactive staining intensity in the cytoplasm and membranes and percentage of stained cells were obtained (see Material and Methods section), and the median score was about 175. In contrast, no OX1R immunodetection was observed in normal tissues, including acinar and ductal cells (FIGS. 2C & 2D). Only three tumors did not show immunoreactivity for OX1R (3/73; 4%). Statistical analyses indicate that OX1R expression is independent of the patient age, gender, disease recurrence, disease-free survival, overall survival, tumor size, TNM stage, lymph node metastasis, and tumor differentiation (not shown). In contrast, no OX1R immunodetection was observed in normal tissue including acinar and ductal cells (FIGS. 2C & 2D).

OX1R Expression in AsPC-1 Human Pancreatic Adenocarcinomas Cell Line

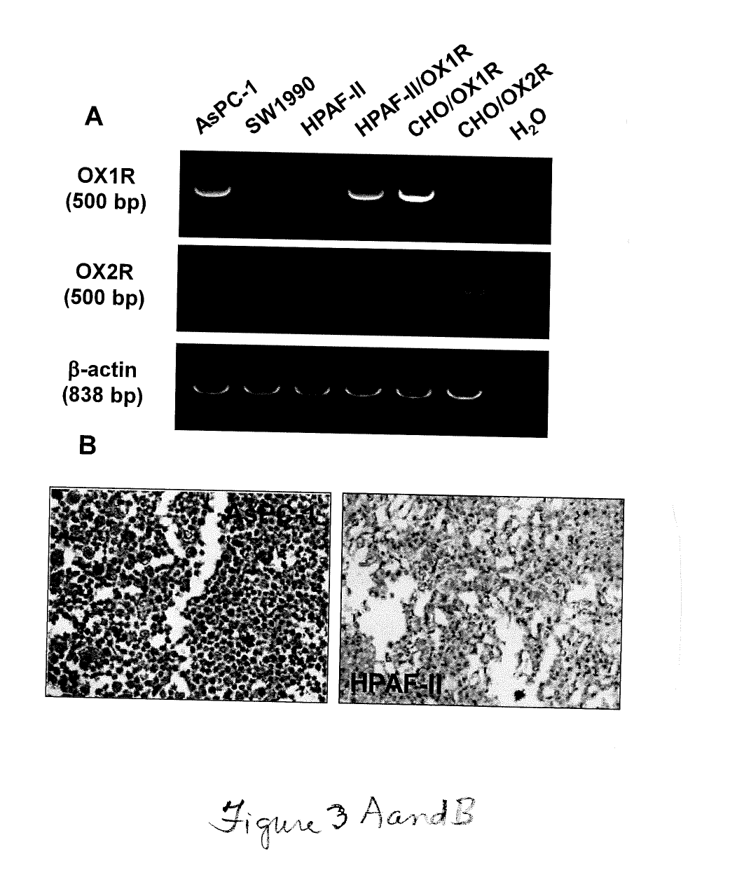

The expression of OX1R was also studied in a large collection of human PDAC cell lines using RT-PCR. As shown in FIG. 3A, an amplified single specific 500 bp PCR product corresponding to OX1R transcript was detected in the AsPC-1 cell line. CHO cells expressing recombinant OX1R receptor were used as control. No OX1R transcript was detected in the SW 1990 and HPAF-II cancer cell lines. As shown in FIG. 3A, no mRNA could be detected for the other orexin receptor subtype, OX2R, in any cell line tested as compared to control recombinant CHO/OX2R cells.

These data are in full agreement with the immunostaining data for OX1R in AsPC-1 and HPAF-II cell lines. Specific OX1R immunodetection was observed in AsPC-1 cell membranes whereas no OX1R expression could be seen in the HPAF-II cell line (FIG. 3B)

Effect of Orexin-A on Pancreatic Cancer Cell Lines

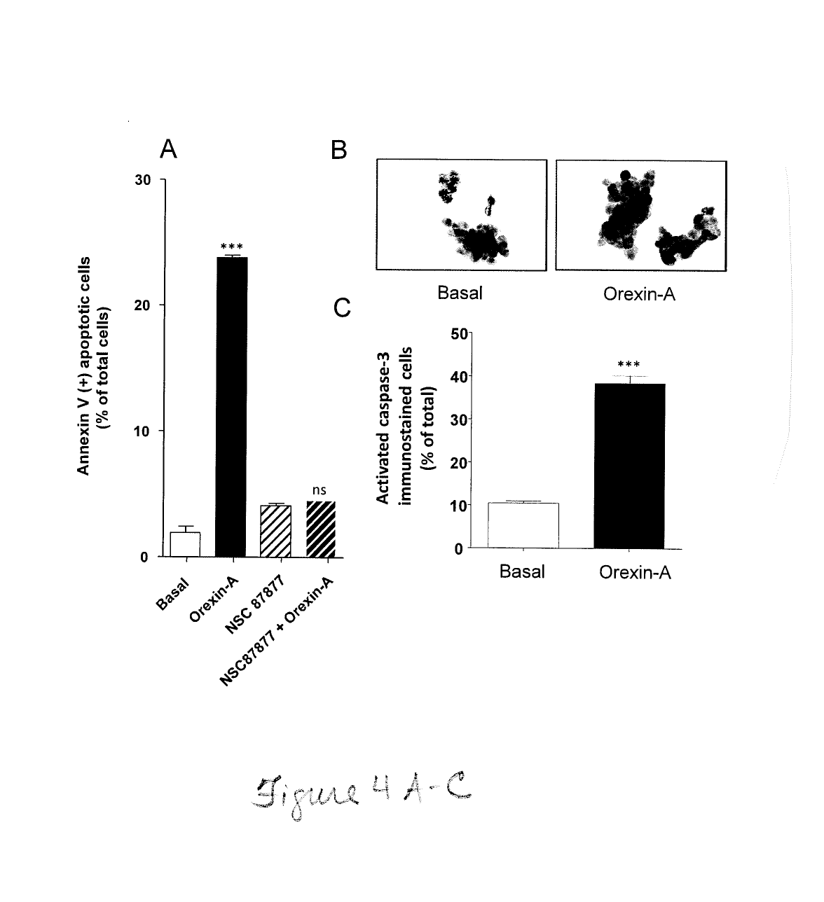

As previously reported in colon cancer cell lines, orexin-A induces a drastic inhibition of cellular growth, associated with the induction of mitochondrial apoptosis, characterized by recruitment of the tyrosine phosphatase SHP-2 and followed by the activation of caspases 3 and 7. Here, we investigated whether orexin-A induced apoptosis in the AsPC-1 cell line. Cells were incubated in the absence or presence of 1 μM orexin-A, and annexin V apoptotic cell staining was quantified after 48 hr. As shown in FIG. 4A, orexin-A induced strong apoptosis in AsPC-1 cells (24.3%±1.4) as compared to untreated cells (3.8%±1.9). In the presence of the specific SHP-2 inhibitor, NSC 87877, orexin-A-induced apoptosis was totally abolished (FIG. 4A), in agreement with the involvement of a SHP-2-dependent apoptosis signaling pathway. No significant difference was observed in the NSC 87877-treated AsPC-1 cells in the presence (4.8%±0.5) or absence of 1 μM orexin-A (4.6%±0.9). Moreover, as seen in FIG. 3B, 1 μM orexin-A induced the cleavage and activation of caspase 3. In contrast, no activated caspase 3 was detected in untreated cells (FIG. 4B). Quantification of activated caspase 3 in AsPC-1 cells demonstrated that orexin-A induced a 4-fold increase in immunostaining as compared to basal conditions (FIG. 4C). Taken together, these results indicate that orexin-A induces apoptosis via recruitment of SHP-2 by OX1R and activation of caspase 3.

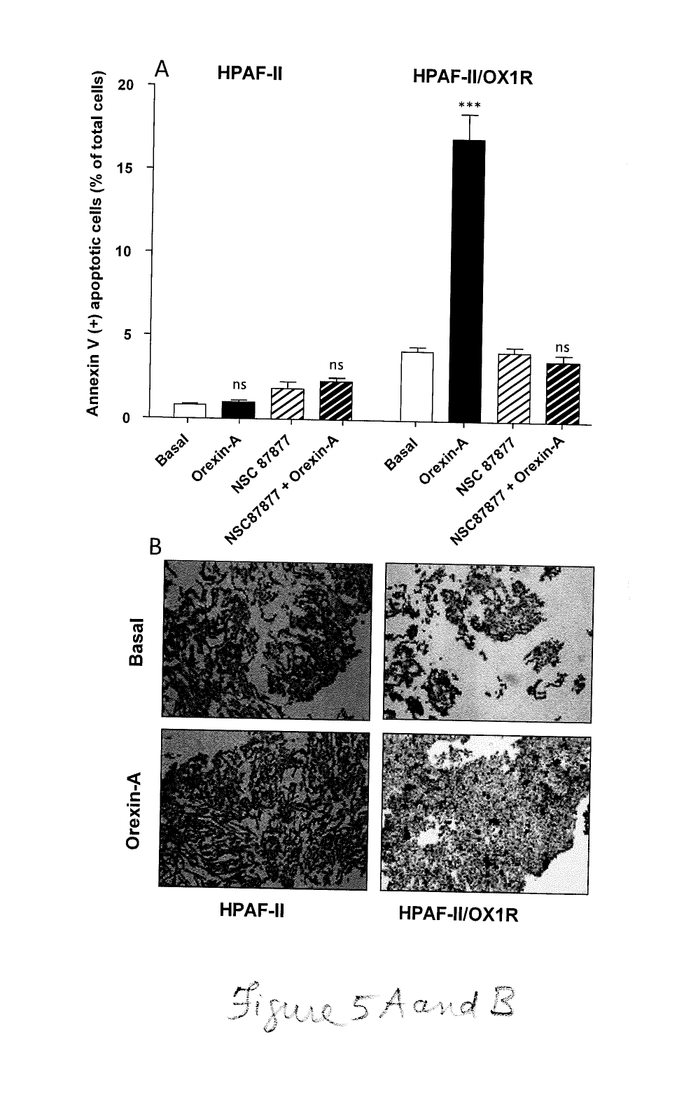

In order to demonstrate the specific proapoptotic role of OX1R in PDAC, we expressed recombinant OX1R in HPAF-II cells, which do not express this receptor. In untransfected parental HPAF-II cells, treatment with 1 μM orexin-A did not induce apoptosis (FIG. 5A). On the contrary, treatment of recombinant HPAF-II expressing OX1R cells with 1 μM orexin-A resulted in the strong induction of cellular apoptosis as indicated by 17.8% 2.4 annexin-V positive apoptotic cells (FIG. 5A) compared to 2.1%±0.4 in untransfected cells. In addition, NSC87877 totally abolished orexin-A-induced apoptosis in OX1R-expressing HPAF-II cells (FIG. 5A), whereas this inhibitor had no effect on the parental cells. Similarly, orexin-A induced strong caspase-3 activation in recombinant OX1R/HPAF-II cells while no activation was observed in the parental cells (FIG. 5B). These data strongly suggest that the proapoptotic role of OX1R is an intrinsic property of the receptor that is not restricted to the AsPC-1 cell context.

Effect of Orexin-A on Growth of Tumors Developed by Xenograft of PDAC Cells in Nude Mice

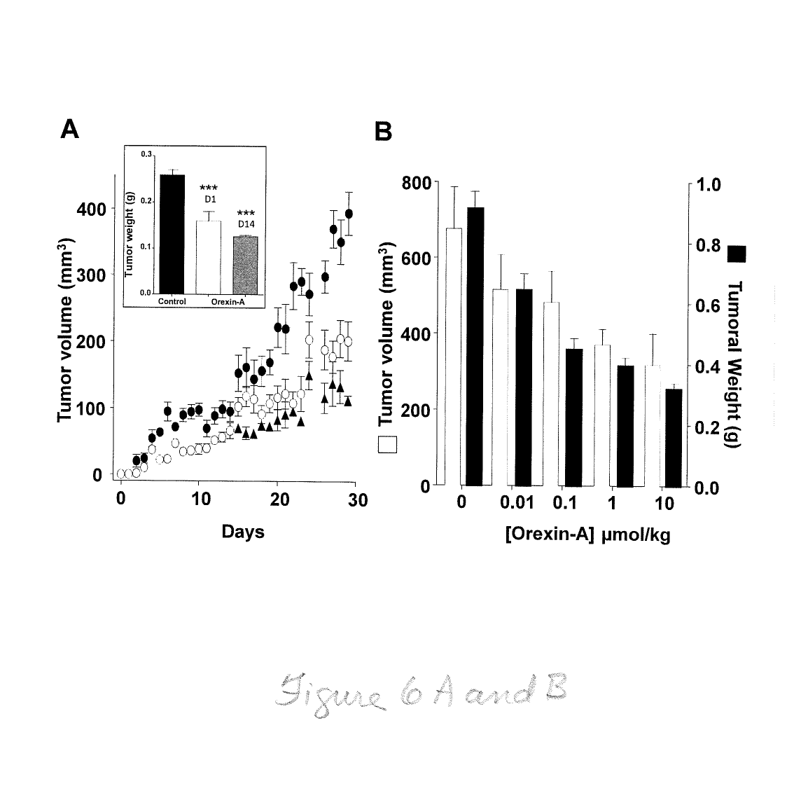

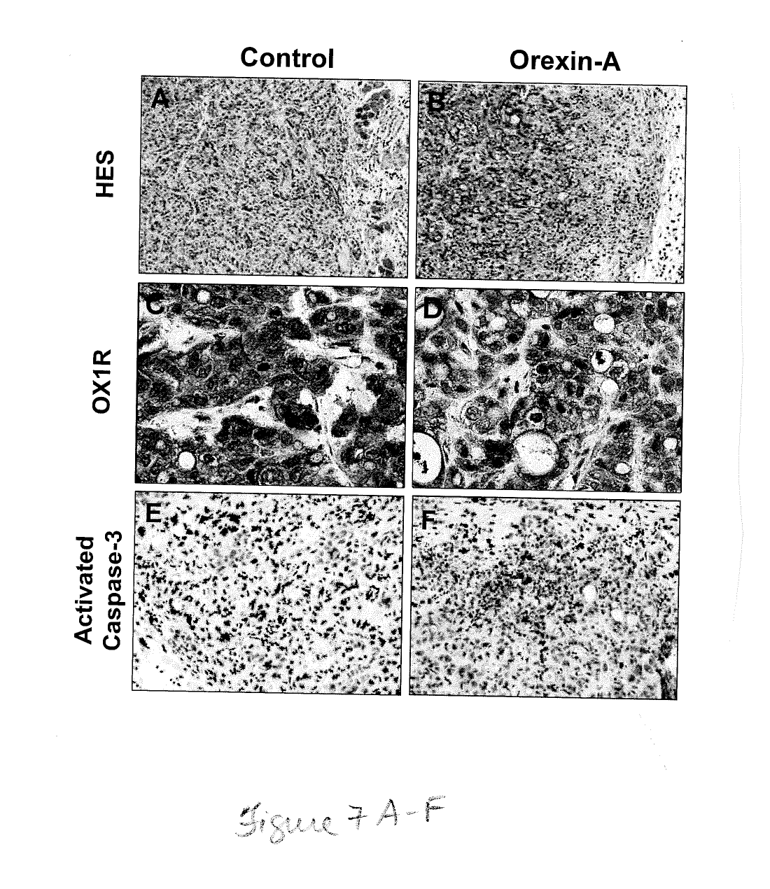

Subcutaneous inoculation of 10⁶ AsPC-1 cells into the flank of nude mice resulted in the development of tumors at the site of inoculation (FIG. 6). Tumor development was followed until 30 or 50 days, and necropsy of mice did not reveal any metastatic sites in any organs such as the pancreas, intestine, colon, liver, spleen, etc. Daily intraperitoneal injection of orexin-A (1 μmol/kg) beginning the day AsPC-1 cells were xenografted into mice and up to the mice sacrifices resulted in a significant decrease in tumor volume (48.8%), as compared to untreated mice (FIG. 6A). The same results were observed under different injection frequencies, i.e., 2 or 3 injections/week (data not shown). In another set of experiments, treatment with orexin-A started after AsPC-1 tumors were established, i.e., 14 days after cell inoculation. Orexin-A (1 μmol/kg), injected, daily, rapidly and strongly reduced the volume of established tumors (FIG. 6A). After animal sacrifice, tumors were resected and weighed. No differences were observed in the weight of tumors from orexin-A treated mice at day 0 and orexin-A treated mice at day 14 after cell inoculation (FIG. 6A, inset). The effect of orexin-A on tumor volume was dose-dependent as a 30-day treatment with 0.01, 0.1, 1 and 10 moles orexin-A/kg decreased the tumor volumes by 34.4, 30.6, 46.7, and 52.8%, respectively (FIG. 6B). It should be noted that 30 day-treatment with and without orexin-A did not affect the weight of mice, i.e., 24.7 g±1.4 g (n=6) and 23.2 g±0.6 g (n=6), respectively. These data correlated with tumoral weight measured after mice were sacrificed (FIG. 6B). Surprisingly, once or twice weekly therapy was equivalent or even more effective in reducing tumor volume than daily injections (data not shown). Hematoxylin and eosin staining of tumors revealed glandular differentiation in both treated and non-treated tumors (FIGS. 7A & 6B). Paraffin sections of AsPC-1 tumors were stained for OX1R and activated caspase-3 (FIG. 7). OX1R immunostaining level was not affected by orexin-A treatment, suggesting that OX1R expression is not altered by chronic treatment (FIGS. 7C & 7D). Furthermore, weak and intense staining of activated caspase-3 was observed in control (FIG. 7E) and orexin-A treated (FIG. 7F) mice, respectively.

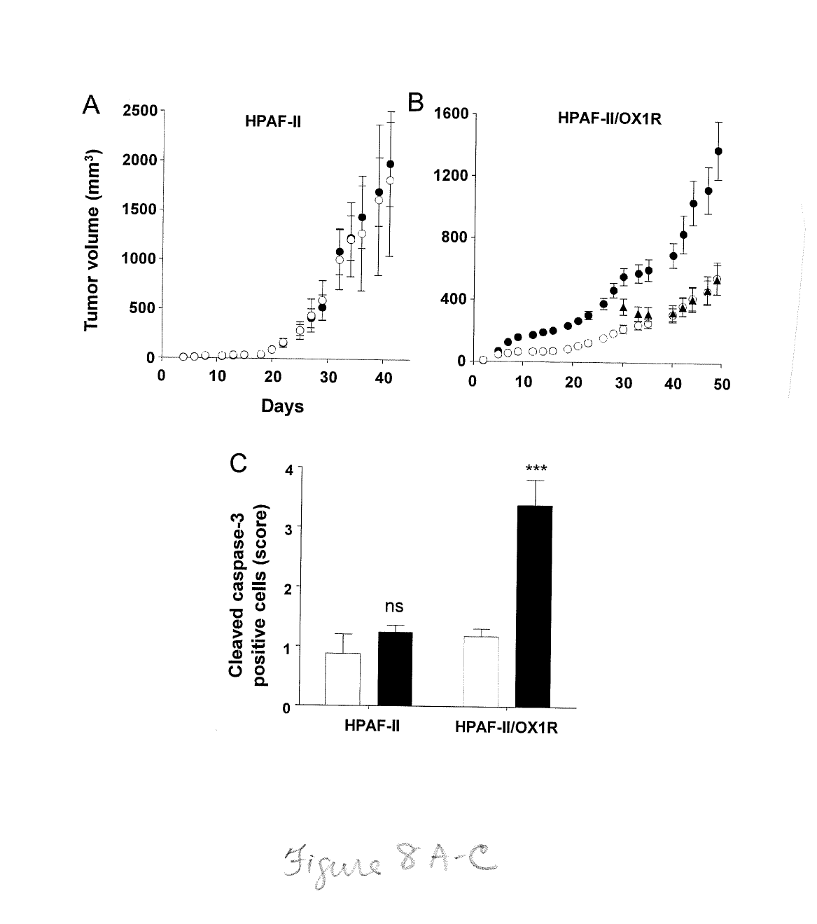

As mentioned above, we demonstrated the specific inhibitory effect of OX1R on tumoral growth in HPAF-II/OX1R xenografted in nude mice. The subcutaneous xenograft in nude mice with the parental HPAF-II or recombinant HPAF-II/OX1R cells resulted in the development of tumors at the site of inoculation (FIG. 8). Daily treatment with 1 mole orexin-A/kg of mice xenografted with parental HPAF-II cells was unable to promote tumor growth inhibition in accordance with the lack of expression of OX1R (FIG. 8A). In contrast, when mice were xenografted with recombinant HPAF-II/OX1R and then treated daily with orexin-A, we observed about 65% inhibition of tumor development (FIG. 8B). Xenografted nude mice treated with orexin-A after 28 days tumor growth showed significant reduction in tumor volumes (FIG. 8B). After animal sacrifices, tumor were resected, fixed, embedded in paraffin and analyzed by IHC. Cleaved caspase-3 positive cells were quantified and scored. FIG. 8C clearly indicates that orexin-A promoted a 3.5-fold caspase-3 activation in tumors from recombinant HPAF-II/OX1R xenografted nude mice as compared to tumors from parental HPAF-II cells. Taken all together, these results demonstrate that the OX1R/orexin-A pathway plays a crucial role in tumor growth inhibition.

In conclusion, data presented in this Example shows that OX1R is aberrantly expressed in most human pancreatic adenocarcinomas, but not in normal cells, and that its activation by exogenous orexins results in strong apoptosis and consequent cell growth inhibition of cancer cells but not of normal cells. In other words, activation of OX1R selectively kills cancer cells. In addition, orexin-A was able to decrease in a dose dependent manner in vivo development of tumors in nude mice xenografted with pancreatic cancer cells, and importantly, to reverse the growth of established tumors. The orexin receptor OX1R thus represents a new specific mediator of apoptosis against pancreatic cancer and a novel candidate for pancreatic cancer therapy.

### Example 3

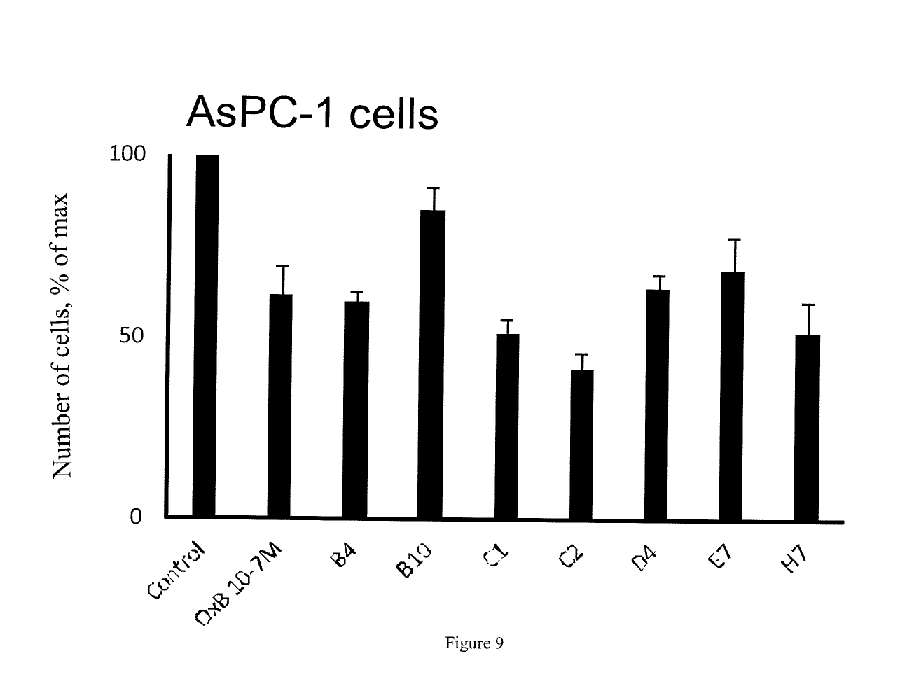

The development of antibodies directed against OX1R were produced by a phage display strategy and the antibody selection was performed by using HEK and HEK stably expressing OX1R (HEK-OX1R) cell lines. As a first step, a batch of 7 different antibodies named B4, B10, C1, C2, D4, E7 and H7 was tested for their ability to inhibit the cell growth of HEK-OX1R. Cells were incubated with 0.1 μM of OxB or antibodies for 48 h in culture medium and then cells were counted in order to estimate the cellular growth. C1 and C2 reduced the HEK-OX1R cell number of about 46%±3 and 37±3% respectively as compared to orexin-B (OxB, 0.1 μM) which reduced of 40±3% the cell number. We have tested the ability of antibodies to inhibit the cellular growth in cancer cell lines derived from and pancreas cancer (AsPC-1 cells). Data reveal that Orexin-B and OX1R antibodies are able to inhibit the cell growth of these cells (FIG. 9).

### REFERENCES

Throughout this application, various references describe the state of the art to which this invention pertains. The disclosures of these references are hereby incorporated by reference into the present disclosure.

1. A method for the treatment of pancreatic cancer in a subject in need thereof comprising administering to the subject a therapeutically effective amount of an OX1R agonist.

2. The method of claim 1 wherein the OX1R agonist is a small organic molecule.

3. The method of claim 1 wherein the OX1R agonist is an antibody.

4. The method of claim 1 wherein the OX1R agonist is selected from the group consisting of chimeric antibodies, humanized antibodies and full human monoclonal antibodies.

5. The method of claim 1 wherein the OX1R agonist is a functional equivalent of Orexin-A or Orexin-B.

6. The method of claim 1 wherein the OX1R agonist is a polypeptide.

7. The method of claim 6 wherein the polypeptide has at least 80% identity with SEQ ID NO: 2 or SEQ ID NO: 3.

8. The method of claim 7, wherein the polypeptide has at least 90% identity with SEQ ID NO:2 or SEQ ID NO:3.

9. The method of claim 8, wherein the polypeptide has a least one substitution, insertion, deletion or amino acid modification relative to SEQ ID NO: 2 or SEQ ID NO: 3.

10. The method of claim 1 wherein the OX1R agonist is an immunoadhesin.

11. The method of claim 1 wherein the OX1R is an aptamer.

12. The method of claim 1 wherein the pancreatic cancer is selected from the group consisting of pancreatic adenocarcinoma, acinar cell cancers, intraductal papillary mucinous neoplasms (IPMN) and pancreatic neuroendocrine tumors.

13. The method of claim 12, wherein said pancreatic adenocarcinoma a pancreatic ductal adenocarcinoma or a serous cystadenoma.

14. The method of claim 12, wherein said pancreatic neuroendocrine tumors is an insulinoma.

15. The method of claim 1 wherein a chemotherapeutic agent is also administered to said subject.

16. A method for treating a pancreatic cancer in a subject in need thereof comprising the steps of
i) determining the expression level of OX1R in a tumour tissue sample obtained from the subject,
ii) comparing the expression level determined at step i) with a reference value and, when the level determined at step i) is higher than the reference value, then
iii) administering to the subject a therapeutically effective amount of an OX1R agonist.

17. A method for screening a drug for the treatment of pancreatic cancer comprising the steps of
i) providing a plurality of test substances;
ii) determining whether the test substances are OX1R agonists; and
iii) positively selecting the test substances that are OX1R agonists.

18. The method of claim 1, wherein said OX1R agonist is one or both of orexin A and orexin B, and wherein the therapeutically effective amount of the OX1R agonist is administered as a bolus, and wherein said step of administering increases a concentration of said orexin A and/or orexin B in said subject to a level that is greater than a normal physiological level.

19. A method of killing human pancreatic cancer cells, comprising
contacting said human pancreatic cancer cells with amount of an OX1R agonist that is sufficient to cause apoptosis of said pancreatic cancer cells, wherein said human pancreatic cancer cells are in vivo.

20. A method of decreasing the size of an established pancreatic cancer tumor in a patient in need thereof, comprising
administering to said patient a therapeutically effective amount of an OX1R agonist.

21. A method of preventing or slowing tumor growth in a patient in need thereof, comprising
administering to said patient a therapeutically effective amount of an OX1R agonist.
